SEQUENCE LISTING (4 sequences)

SEQ ID NO: 1 - protein, 187 aa, Bordetella pertussis
  REGION 1..187: note = Description of sequence: BP0205 protein
MQLTIRKLAYTLAFSTLVLAGCTTASKKTDGQAATPADQASSQQASAASVEFYVAQAKAGDGLMEVKVPDGSLYMQRQPVLTRADLTEAAALVDRQGQNFVGLRFTEAGARKLNDISSKNIGNMLALVIDRELVAAPRIAEPLNRGVLAFGVPSAKAASEIAAKIRGDAGAPAAGVPAAPAPKPAPK

SEQ ID NO: 2 - protein, 15 aa, Bordetella pertussis
  REGION 1..15: note = Description of sequence: Antigen epitope
IDRELVAAPRIAEPL

SEQ ID NO: 3 - protein, 16 aa, Bordetella pertussis
  REGION 1..16: note = Description of sequence: Antigen epitope
GVLAFGVPSAKAASEI

SEQ ID NO: 4 - protein, 22 aa, Bordetella pertussis
  REGION 1..22: note = Description of sequence: Signal peptide
MQLTIRKLAYTLAFSTLVLAGC

(19) United States
(12) Patent Application Publication
(10) Pub. No.: US 2026/0083833 A1
(43) Pub. Date: Mar. 26, 2026

(54) IMMUNOGENIC PROTEINS FROM BORDETELLA PERTUSSIS

(71) Applicant: OHIO STATE INNOVATION FOUNDATION, Columbus, OH (US)

(72) Inventor: Purnima DUBEY, Columbus, OH (US)

(21) Appl. No.: 19/111,155

(22) PCT Filed: Sep. 15, 2023
(86) PCT No.: PCT/US2023/074302

§ 371 Date: Mar. 12, 2025

Related U.S. Application Data
(60) Provisional application No. 63/408,244, filed on Sep. 20, 2022.

(51) Int. Cl. A61K 39/02; A61P 37/04

(52) U.S. Cl. CPC A61K 39/099; A61P 37/04

(57) ABSTRACT

Recent evidence accumulating over the last decade demonstrates that generation of CD4+ T cells is critical for sustained immunity against Bordetella pertussis. B. pertussis contains hundreds of antigens that are processed and presented on MHC Class II and recognized by CD4′T cells. The present disclosure relates to a vaccine comprising Bordetella pertussis antigen peptides to prevent infection of the Bordetella pertussis bacterium.

### CROSS REFERENCE TO RELATED APPLICATIONS

This application claims priority to, and the benefit of, U.S. Provisional Patent Application No. 63/408,244, filed Sep. 20, 2022, entitled “IMMUNOGENIC PROTEINS FROM BORDETELLA PERTUSSIS,” which is incorporated by reference herein in its entirety.

### STATEMENT REGARDING FEDERALLY SPONSORED RESEARCH

This invention was made with government support under grant number A1153829, awarded by the National Institutes of Health (NIH). The government has certain rights in the invention.

### REFERENCE TO SEQUENCE LISTING

The sequence listing submitted on Sep. 15, 2023, as an .XML file entitled “103361-361WO1.xml” created on Sep. 15, 2023, and having a file size of 6,384 bytes is hereby incorporated by reference pursuant to 37 C.F.R. § 1.52(e)(5).

### FIELD

The present disclosure relates to a vaccine comprising Bordetella pertussis antigen peptides to prevent infection of the Bordetella pertussis bacterium.

### BACKGROUND

Recent evidence accumulating over the last decade demonstrates that generation of CD4+ T cells is critical for sustained immunity against Bordetella pertussis (B. pertussis, the bacterium that causes the disease pertussis). B. pertussis contains hundreds of antigens that are processed and presented on MHC Class II (HLA-DR) and recognized by CD4+ T cells. However, there is a need for next generation pertussis vaccines that elicit strong, long-lived systemic and mucosal immunity against B. pertussis. The current acellular pertussis vaccines (aPV) contain 3-5 proteins adjuvanted with alum. These proteins were selected for their roles in pathogenesis of B. pertussis with the goal of generating neutralizing antibodies that remove the bacteria from the host. However, the efficacy of these vaccines are not long lasting.

Given the limitations described above, there is a need to identify novel B. pertussis proteins to incorporate into a vaccine thus leading to development of novel vaccine therapies to generate better and longer lasting immune responses.

The compositions, and methods disclosed herein address these needs.

### SUMMARY

The present disclosure provides vaccine compositions and methods of use thereof to prevent and elicit an immune response against B. pertussis infections.

In one aspect, disclosed herein is a pertussis vaccine comprising a BP0205 antigen, or variants thereof, and at least one peptide from a Bordetella pertussis (B. pertussis) bacterium, wherein the BP0205 peptide and the at least one peptide are to be presented to an MHC Class II cell and recognized by a CD4+ T cell.

In some embodiments, the BP0205 antigen comprises at least 70% sequence identity to SEQ ID NO: 1. In some embodiments, the BP0205 antigen comprises at least 80% sequence identity to SEQ ID NO: 1. In some embodiments, the BP0205 antigen comprises at least 90% sequence identity to SEQ ID NO: 1. In some embodiments, the BP0205 antigen comprises SEQ ID NO: 1.

In some embodiments, the vaccine comprises an antigen epitope comprising SEQ ID NO: 2 or SEQ ID NO: 3. In some embodiments, the vaccine comprises an antigen epitope comprising SEQ ID NO: 2 and SEQ ID NO: 3.

In some embodiments, the at least one peptide originates from a protein involved in at least one biological pathway within the B. pertussis bacterium. In some embodiments, the at least one peptide originates from a protein of Table 2 which comprises B. pertussis proteins of interest.

In some embodiments, the at least one pathway is selected from cell adhesion and motility, cell cycle, cell signaling, cell structure, cell transport, gene regulation, metabolism, oxidation-reduction, protein regulation, or stress responses.

In some embodiments, the protein selected from cell adhesion and motility pathways comprise attaching and effacing protein, fimbrial protein, flagellar biosynthesis protein (FlhF), or combinations thereof.

In some embodiments, the protein selected from cell cycle pathways comprise beta-lactamase domain-containing protein, cell division coordinator (CpoB), cell division protein (ZapD), peptidyl-prolyl cis-trans isomerase, peptidyl-prolyl cis-trans isomerase (Cbf2), phospho-N-acetylmuramoyl-pentapeptide-transferase, tol-Pal system protein (TolB), tol-Pal system protein (TolQ), or combinations thereof.

In some embodiments, the protein selected from cell signaling pathways comprise dermonecrotic toxin, cyclic di-GMP phosphodiesterase response regulator (RpfG), enterobactin outer-membrane receptor, or combinations thereof.

In some embodiments, the protein selected from cell structure pathways comprise metal-binding protein, BrkB, D-alanine-D-alanine ligase, inner membrane protein (ybhN), inner membrane protein (YiaH), lipoprotein, lipoteichoic acid synthase 2, outer membrane protein assembly factor (BamA), P.93, penicillin-binding protein 1A, pertussis toxin liberation protein F, membrane protein, DMT superfamily permease, spermidine synthase with an N-terminal membrane domain, lipoate-protein ligase A, HflK, assembly protein, inner membrane protein, HI_0719, soluble lytic murein transglycosylase, virulence-associated outer membrane protein Vir-90, or combinations thereof.

In some embodiments, the protein selected from cell transport pathways comprise Xaa-Pro aminopeptidase, 2-aminoethylphosphonate ABC transporter substrate-binding protein, aerobactin synthase (IucC), antiseptic resistance protein, arabinose efflux permease, BFD-like [2Fe-2S] binding domain, bicarbonate transport ATP-binding protein (CmpD), bicyclomycin/multidrug efflux system, biotin transporter, BrkA autotransporter, ComEC family competence protein, D-methionine-binding lipoprotein (metQ), drug efflux system protein (MdtG), efflux pump membrane transporter, extracytoplasmic solute receptor protein, extracytoplasmic solute receptor protein (yiaO), ferrichrome receptor (FcuA), filamentous hemagglutinin, heme-repressible hemoglobin-binding protein, hemin import ATP-binding protein (HmuV), inner membrane metabolite transport protein (yhjE), inner membrane protein (yhjX), inner membrane transport protein (ydhP), iron-sulfur cluster carrier protein, leucine ABC transporter subunit substrate-binding protein (LivK), leucine-, isoleucine-, valine-, threonine-, and alanine-binding protein, LIV-I protein F, LIV-I protein H, major facilitator superfamily proteins, manganese transport system membrane protein (mntB), methyl viologen resistance protein (SmvA), multidrug export ATP-binding/permease protein (SAV1866), multidrug resistance protein B, multiple resistance and pH homeostasis protein A, multiple resistance and pH homeostasis protein D, multiple resistance and pH homeostasis protein E, Neu5Ac permease, nickel/cobalt efflux system, BP0840, preprotein translocase subunit (SecD), ABC transporter ATP-binding protein (HI_0664), amino-acid metabolite efflux pump, chorismate pyruvate-lyase, membrane transporter protein, protein translocase subunit (SecD), purine efflux pump (PbuE), putrescine transport system permease protein (PotH), RTX-I toxin determinant B, sec-independent protein translocase protein (TatC), short-chain fatty acids transporter, sn-glycerol-3-phosphate transport system permease protein (ugpA), spermidine/putrescine transport system permease protein (PotB), TonB family C-terminal domain, TpsB transporter, transport permease protein, TRAP-type C4-dicarboxylate transport system, small permease component, TRAP-type uncharacterized transport system, fused permease components, tripartite tricarboxylate transporter (TctA), or combinations thereof.

In some embodiments, the protein selected from gene regulation pathways comprise type IV secretion system protein virB11, 30S ribosomal protein S10, 50S ribosomal protein L10, 50S ribosomal protein L14, 50S ribosomal protein L25, 50S ribosomal protein L31 type B, ATP-dependent DNA helicase Rep, ATP-dependent RNA helicase (HrpA), bacterial DNA-binding protein, BpH3, cyn operon transcriptional activator, DNA polymerase III subunit gamma/tau, DNA protection during starvation protein 2, DNA repair protein (RecN), galactose-binding protein regulator, Gev operon activator, HTH-type transcriptional regulatory protein (gabR), leucine-responsive regulatory protein, LigA, polyribonucleotide nucleotidyltransferase, protein (ApaG), ribonuclease (TTHA0252), ribosomal RNA small subunit methyltransferase B, ribosome-binding factor A, ribosome-recycling factor, single-stranded DNA-binding protein, transcriptional regulator, y4mF family, transcription-repair-coupling factor, or combinations thereof.

In some embodiments, the protein selected from metabolic pathways comprise ACR, COG1565, 1,4-alpha-glucan branching enzyme (GlgB), 1,4-dihydroxy-2-naphthoyl-CoA synthase, 2-(hydroxymethyl) glutarate dehydrogenase, 2,3,4,5-tetrahydropyridine-2,6-dicarboxylate N-acetyltransferase, 3-(3-hydroxy-phenyl) propionate/3-hydroxycinnamic acid hydroxylase, 3-hydroxyacyl-CoA dehydrogenase, 3-oxoadipate enol-lactonase 2, 3-oxosteroid 1-dehydrogenase, 4-aminobutyrate aminotransferase PuuE, 4-hydroxy-3-methylbut-2-en-1-yl diphosphate synthase (flavodoxin), 5-formyltetrahydrofolate cyclo-ligase, 5-methyltetrahydropteroyltriglutamate—homocysteine methyltransferase, 6-aminohexanoate-cyclic-dimer hydrolase acetaldehyde dehydrogenase 2, acetolactate synthase isozyme 3 large subunit, acetone carboxylase alpha subunit, acetone carboxylase beta subunit, acetyl-CoA acetyltransferase, acetyltransferase component of pyruvate dehydrogenase complex, acyl carrier protein, acyl-[acyl-carrier-protein]-UDP-N-acetylglucosamine O-acyltransferase, acylase (ACY 1), acyl-CoA dehydrogenase (AidB), short-chain specific acyl-CoA dehydrogenase, adenosylhomocysteinase, aldehyde dehydrogenase, thermostable aliphatic sulfonates transport permease protein (ssuC), amidophosphoribosyltransferase, aminotransferase, arginine transport ATP-binding protein (ArtM), argininosuccinate lyase, aspartate 1-decarboxylase, aspartoacylase, aspartokinase, ATP synthase subunit beta, ATP-dependent dethiobiotin synthetase (BioD), beta-ketoacyl-acyl-carrier-protein synthase 1, biotin carboxyl carrier protein of acetyl-CoA carboxylase, biotin carboxylase, carbamoyl-phosphate synthase large chain, carbamoyl-phosphate synthase small chain, cytochrome c, cytochrome c oxidase subunit 2, D-amino acid dehydrogenase, dihydrolipoyl dehydrogenase, dihydrolipoyllysine-residue succinyltransferase component of 2-oxoglutarate dehydrogenase complex, dihydropteroate synthase, D-malate degradation protein R, D-malate dehydrogenase (decarboxylating), exported protein, ferredoxin-dependent glutamate synthase 1, Flp pilus assembly protein (TadD), formate-dependent phosphoribosylglycinamide formyltransferase, formyl-coenzyme A transferase, glutamate dehydrogenase, glutamine—fructose-6-phosphate aminotransferase, glyceraldehyde-3-phosphate dehydrogenase, glycine dehydrogenase, guanidinobutyrase, imidazoleglycerol-phosphate dehydratase, iron-sulfur cluster assembly scaffold protein (IscU), L-aspartate dehydrogenase, long-chain-fatty-acid—CoA ligase, malate dehydrogenase, maltose alpha-D-glucosyltransferase, MmgE/PrpD family, N5-carboxyaminoimidazole ribonucleotide synthase, NAD⁺ binding domain of 6-phosphogluconate dehydrogenase, nicotinamidase/pyrazinamidase, nucleoside recognition, Oxoglutarate dehydrogenase, phosphoserine aminotransferase, hydrolase of the alpha/beta-hydrolase fold, protoheme IX biogenesis protein, soluble aldose sugar dehydrogenase (yliI), sporulation inhibitor (kipI), stringent starvation protein A, succinate dehydrogenase flavoprotein subunit, succinate-semialdehyde dehydrogenase [NADP(+)] (GabD), thiamine biosynthesis protein (HI_0357), thiosulfate sulfurtransferase, threonine synthase, thymidylate synthase, UDP-glucose 6-dehydrogenase, Vi polysaccharide biosynthesis protein (TviD), xanthine dehydrogenase accessory protein (XdhC), or combinations thereof.

In some embodiments, the protein selected from oxidation-reduction pathways comprise 3-alpha-(Or 20-beta)-hydroxysteroid dehydrogenase, 3-ketosteroid-9-alpha-hydroxylase reductase subunit, 3-oxoacyl-[acyl-carrier-protein] reductase (FabG), acetoin: 2,6-dichlorophenolindophenol oxidoreductase subunit beta, alkyl hydroperoxide reductase C, benzaldehyde dehydrogenase [NAD(+)], benzene 1,2-dioxygenase subunit alpha, catalase, dTDP-4-dehydrorhamnose reductase, gamma-glutamylputrescine oxidoreductase, gluconate 2-dehydrogenase cytochrome c subunit, glutathione hydrolase proenzyme, glutathione import ATP-binding protein (GsiA), glutathione transport system permease protein (gsiD), glutathione-binding protein (gsiB), glyoxylate/hydroxypyruvate reductase A, glyoxylate/hydroxypyruvate reductase B, heme A synthase, L-2-hydroxyglutarate oxidase (LhgO), methylmalonate-semialdehyde dehydrogenase, Mg-chelatase subunit child, muconolactone delta-isomerase, NAD(P)H-hydrate epimerase, NADH-quinone oxidoreductase, NADH-quinone oxidoreductase subunit L, NADH-quinone oxidoreductase subunit M, NADH-quinone oxidoreductase subunit N, aldo-keto reductase, NAD(P)H quinone oxidoreductase, PIG3, quinone oxidoreductase 1, rhodocoxin reductase, sarcosine oxidase subunit beta, sulfite reductase, thiol:disulfide interchange protein (DsbD), thioredoxin reductase, ubiquinol oxidase subunit 1, or combinations thereof.

In some embodiments, the protein selected from protein regulation pathways comprise apolipoprotein N-acyltransferase, ATP-dependent Clp protease ATP-binding subunit (ClpX), capsule polysaccharide biosynthesis protein, carboxypeptidase G2, chaperonin GroEL, chorismate synthase, co-chaperonin GroES, dihydroxy-acid dehydratase, FtsH protease regulator (HflK), small chain glutamate synthase, glutamine synthetase, lon protease, membrane dipeptidase M19, membrane protein insertase (YidC), methionine-tRNA ligase, molybdopterin molybdenumtransferase, oligopeptidase A, periplasmic serine endoprotease DegP-like, C-terminal PDZ domain protease, HtpX, protein-L-isoaspartate O-methyltransferase, glutamate—cysteine ligase 2, tRNA pseudouridine synthase A, tryptophan-tRNA ligase, tyrosine-tRNA ligase, or combinations thereof.

In some embodiments, the protein selected from stress response pathways comprise Rv1996/MT2052, SAV1710, or combinations thereof.

In some embodiments, the pertussis vaccine further comprises any combination of adjuvants, preservatives, or stabilizers. In some embodiments, the vaccine comprises a pharmaceutically acceptable carrier selected from an excipient, a diluent, a salt, a buffer, a stabilizer, a lipid, an emulsion, or a nanoparticle.

In one aspect, disclosed herein is a method of preventing an infection from a Bordetella pertussis (B. pertussis) bacterium in a subject, the method comprising administering to the subject a pharmaceutically effective amount of the pertussis vaccine of any preceding aspect.

In one aspect, disclosed herein is a method of improving an immune response against a Bordetella pertussis (B. pertussis) bacterium in a subject, the method comprising administering to the subject a pharmaceutically effective amount of the pertussis vaccine of any preceding aspect.

In some embodiments, the infection from the Bordetella pertussis bacterium causes whooping cough in the subject. In some embodiments, the immune response comprises presenting the antigen to an MHC Class II cell, recognition of the antigen by a CD4+ T cell, or combination thereof. In some embodiments, the immune response is against a BP0205 antigen and at least one protein involved in at least one biological pathway within the B. pertussis bacterium. In some embodiments, the at least one pathway selected from cell adhesion and motility, cell cycle, cell signaling, cell structure, cell transport, gene regulation, metabolism, oxidation-reduction, protein regulation, or stress responses.

In some embodiments, the method further comprises administering the pertussis vaccine through intranasal, intramuscular, intraperitoneal, or subcutaneous routes of injection.

In some embodiments, the subject is a mammal. In some embodiments, the subject is a human.

### BRIEF DESCRIPTION OF FIGURES

The accompanying figures, which are incorporated in and constitute a part of this specification, illustrate several aspects described below.

FIG. 1 shows the immunoprecipitation of MHC-II epitopes.

FIGS. 2A and 2B show the IFNγ secretion by splenocytes stimulated with target peptides was evaluated by ELISA. Splenocytes from either naive (FIG. 2A) or convalescent mice (FIG. 2B) were stimulated with peptides or media alone as no stimulation (NS) negative control. One-way ANOVA with Tukey's multiple comparisons was used to detect differences between all experimental groups. Significance is indicated above for each group (*p<0.05; **p<0.01: ***p<0.001; ****p<0.0001). Each dot represents an individual well. Data are displayed as mean and SEM.

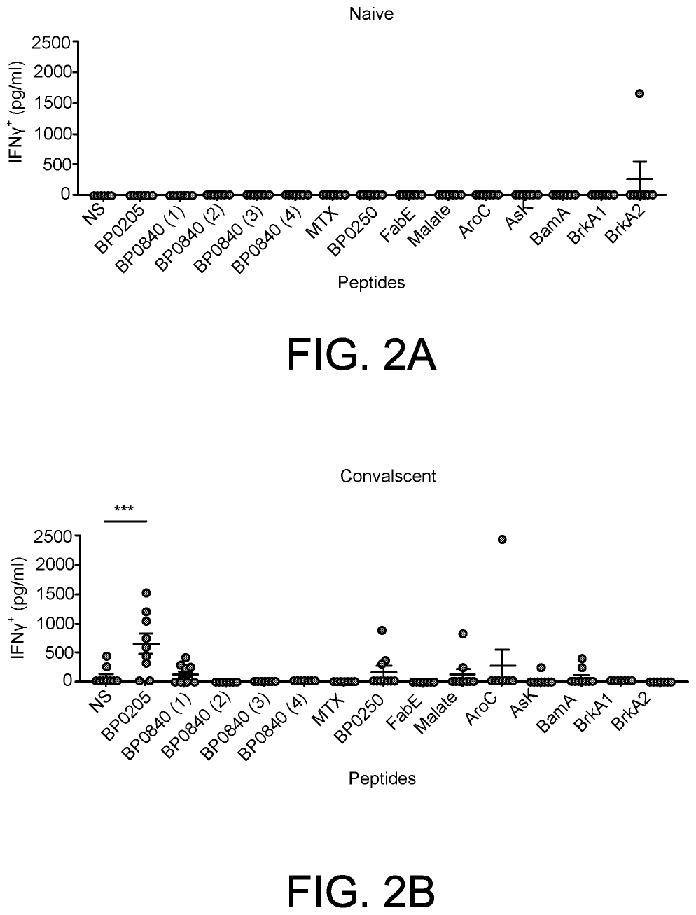

FIGS. 3A and 3B show the cell proliferation assay using 3H Thymidine incorporation. Target peptides identified from our screening were used to stimulate splenocytes from either naive mice (FIG. 3A) or mice immunized with heat killed bacteria (FIG. 3B). Media alone was used as no stimulation (NS) negative control. Significance is indicated above for each group (*p<0.05; **p<0.01; ***p<0.001, ****p<0.0001). Each dot represents an individual well. Results are displayed as mean and SEM.

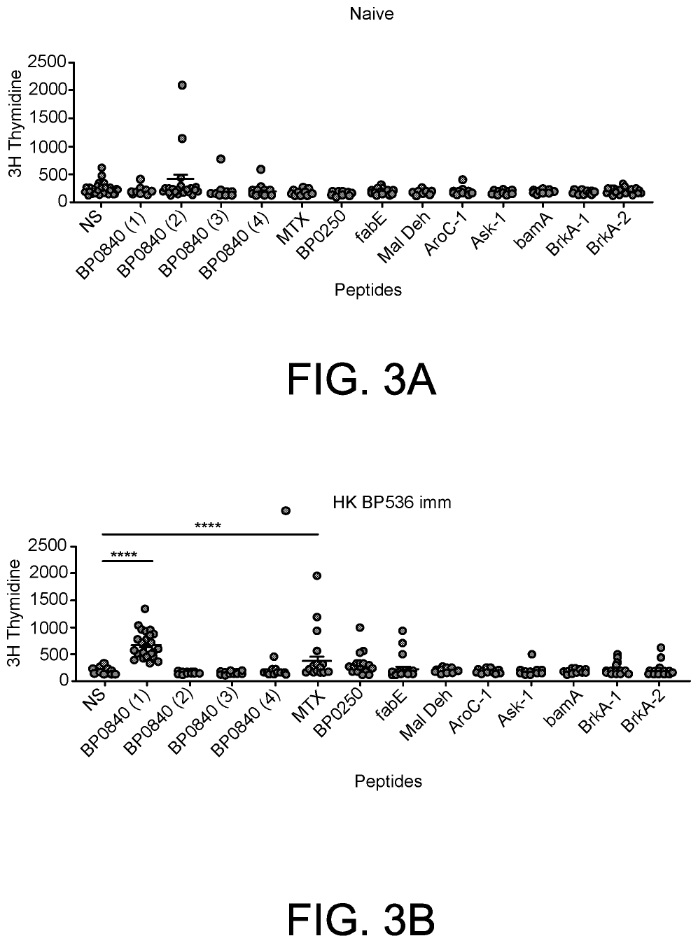

FIG. 4 shows the human AIM gating strategy.

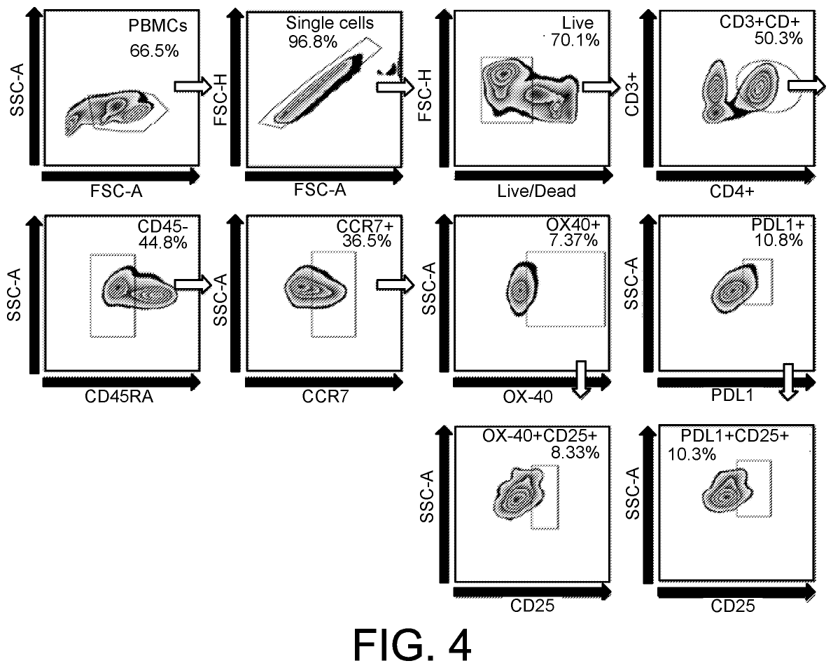

FIG. 5 shows the Bp0840 and Bp0205 stimulate expression of early activation markers by T cells of DTP primed Tdap boosted patients. Promising peptides identified from our screening were used to stimulate human PBMCs isolated from vaccinated donors. T cell stimulation was evaluated using flow cytometry by comparing expression of activation induced markers (AIM). Media alone was used as no stimulation (−) negative control. Each symbol is an individual participant (7 per group).

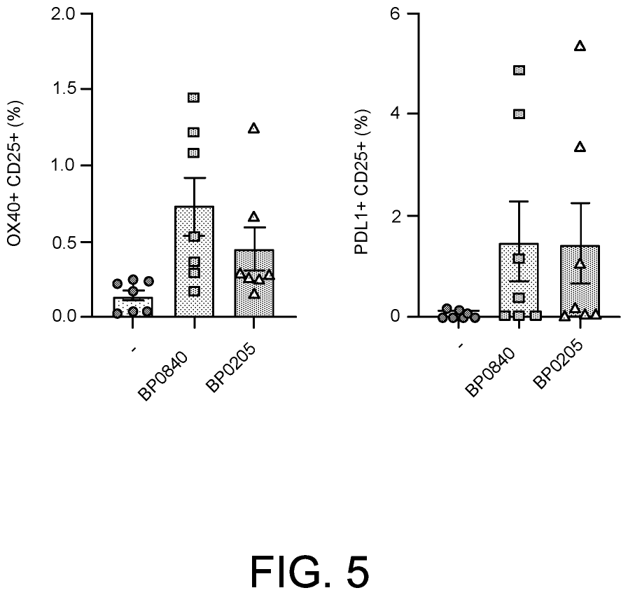

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Enhance” can refer to any change that results in a greater amount of a symptom, disease, composition, condition, response, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% improvement so long as change is statistically significant.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

By “reduce” or other forms of the word, such as “reducing” or “reduction,” is meant lowering of an event or characteristic (e.g., bacterial infection). It is understood that this is typically in relation to some standard or expected value, in other words it is relative, but that it is not always necessary for the standard or relative value to be referred to. For example, “reduces bacterial infection” means reducing the rate of bacterial growth and spread within a subject relative to a standard or a control.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient.

The term “pharmaceutically effective amount” refers to the amount of the composition used is of sufficient quantity to ameliorate one or more causes or symptoms of a disease or disorder. Such amelioration only requires a reduction or alteration, not necessarily elimination.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, the term “infection” refers to the invasion of tissues by pathogens, their multiplication, and reaction of host tissues to the infectious agent and any toxins they release Infections can be caused by a wide range of pathogen, most common are bacteria and viruses.

A “protein,” “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods consider conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md, at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

It should also be noted that amino acids, and derivatives (with exception of glycine) occurs in two isomeric forms: L-forms or D-forms. The L- and D-forms represent the same atoms of an amino acid, however the atoms can have different arrangements, which can impact the amino acid properties and functions. The two forms are similar in that they both occur naturally and comprise a central carbon atom, at least one hydrogen atom, a carboxylic group, an amine group, and a variable group. The two forms differ in that they are usually mirrored images of each other, wherein the location of the amine group varies. L-amino acids are used in protein synthesis, while D-amino acid are less common in protein synthesis. L-amino acids rotate counterclockwise or to the left in a process known as levorotation. D-amino acids rotate clockwise or to the right in a process known as dextrorotation. L-amino acids are used to synthesize proteins, while D-amino acids are found in the cell walls of bacteria.

The term “variant” means a polypeptide derived from a parent albumin by one or more (several) alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X,’ the insertion may be at position ‘X+1’ (‘downstream’) or at position ‘X−1’ (‘upstream’).

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

A variant polypeptide may have substantially the same functional activity as a reference polypeptide. For example, a variant polypeptide may exhibit or more biological activities associated with binding a ligand.

Variants comprising a fragment of a reference amino acid sequence are contemplated herein. A “fragment” is a portion of an amino acid sequence which is identical in sequence to but shorter in length than the reference sequence. A fragment may comprise up to the entire length of the reference sequence, minus at least amino acid residue. For example, a fragment may comprise from 5 to 1000 or contiguous amino acid residues of a reference polypeptide. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous amino acid residues of a reference polypeptide, respectively. Fragments may be preferentially selected from certain regions of a molecule, for example the N-terminal region and/or the C-terminal region of a polypeptide. The term “at least a fragment” encompasses the full length polypeptide.

A “vaccine” refers to a biological preparation that provides active acquired immunity to a particular infectious disease caused by a virus, bacteria, parasite, or any other microorganism Vaccines typically comprise an agent or several agents, also referred to as antigens, that are expressed by the disease-causing microorganism and are often made from weakened or killed forms of the microbe, its toxins, or its surface proteins/peptides. Vaccines are also made to comprise additional components, such as adjuvants, preservatives, and/or stabilizers to boost the immune response, improve safety, and improve vaccine storage.

An “antigen” refers to a molecule, moiety, foreign particulate matter, or an allergen that can bind to a specific antibody or T cell receptor. The presence of antigens within a host can illicit an immune response against said molecule, moiety, foreign particulate matter, or allergen.

An “adjuvant” refers to a drug, molecule, substance, or a combination thereof that is used to increase the efficacy or potency of certain therapeutic agents, such as for example vaccines and/or antibodies. “Adjuvant(s)” are often at least one ingredient used in some vaccines that help create a stronger immune response in the host receiving said vaccine.

The terms “cell,” “cell line” and “cell culture” include progeny. It is also understood that all progenies may not be precisely identical in DNA content, due to deliberate or inadvertent mutations. Variant progeny that have the same function or biological property, as screened for in the originally transformed cell, are included. The “host cells” used in the present invention generally are prokaryotic or eukaryotic hosts.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compounds and Compositions

pertussis vaccines are a type of vaccine to protect against the Bordetella pertussis bacterium known to cause “whooping cough” in humans. There are two types of pertussis vaccines: whole-cell vaccines and acellular vaccines. The acellular vaccine type protects the respiratory tract with fewer side effects compared to whole-cell pertussis vaccine; however, the efficacy of acellular pertussis vaccines declines at a much faster rate, and acellular vaccines do not elicit clearance of the upper respiratory tract. Given the limitations of current pertussis vaccines, there is a need to produce an improved pertussis vaccine with increased efficacy and long-lasting protection.

B. pertussis bacterium are known to infect the upper and lower respiratory tract leading to an immune response. Infection by this bacterium induces both innate (neutrophil and macrophage immune cells) and adaptive (B and T cell) immune responses. In general, antigen-presenting cells such as dendritic cells, macrophages, and some endothelial cells can present B. pertussis peptides on their cell surface bound to major histocompatibility complex II (MHCII) in mice and HLA-DR in people. This recognition activates CD4+ T cell responses, a component of the adaptive immune response. The presentation of B. pertussis peptides of the immune cell surface is then recognized by T cells, such as CD4+ T cells. Increased numbers of CD4+ T cells lead to B. pertussis clearance from the respiratory organs.

Herein, an unbiased biochemical approach was used to identify peptides from B. pertussis that are presented on MHC Class II. These peptides are derived from various B. pertussis proteins that are not part of the current acellular pertussis vaccines. The experimental evidence supports the immunogenicity of these peptides and shows that inclusion of these novel proteins in a next generation pertussis vaccine generate better and longer lasting immune responses than the current vaccines.

The present disclosure relates to an improved vaccine against B. pertussis bacterial infection comprising novel proteins expressed by B. pertussis that are presented on HLA-DR and MHC Class II molecules as peptides.

In one aspect, disclosed herein is a pertussis vaccine comprising a BP0205 antigen, or variants thereof, in combination with at least one additional peptide from a B. pertussis bacterium, wherein the BP0205 peptide and the at least one additional peptide are to be presented to an MHC Class II cell and recognized by a CD4+ T cell.

In some embodiments, the pertussis vaccine comprises an acellular pertussis (ap) vaccine.

In some embodiments, the at least one peptide originates from a protein involved in at least one biological pathway within the B. pertussis bacterium. In some embodiments, the at least one peptide originates from a protein of Table 2 which comprises B. pertussis proteins of interest.

The vaccine targets, or antigens, are identified and validated experimentally for commercial interest of vaccine manufacturers who are working on improving the efficacy of pertussis vaccines. Further the antigens are intracellular and extracellular components of the B. pertussis bacterium. Herein, the antigens comprise cell surface proteins, lipopolysaccharides, peptidoglycans, and intracellular proteins critical for bacterial gene regulation, bacterial protein regulation, bacterial metabolism, bacterial cell cycle, bacterial cell adhesion and motility, and bacterial cell stress responses.

The present disclosure provides a pertussis vaccine comprising a BP0205 peptide or protein as an antigen. BP0205 is a lipoprotein or lipopeptide comprising a signal peptide (exemplified in SEQ ID NO: 4), and is found in the inner membrane of B. pertussis bacteria. BP0205 is contemplated to be involved in the insertion, folding, and complex formation of integral membrane proteins into the cell membrane. By “BP0205 peptide” or “BP0205 protein” is meant the entire peptide, or a fragment thereof which is recognized as an antigen. One of skill in the art will understand that variants, fragments, or portions of the BP0205 peptide can be used as an antigen. One can measure the antigenic response of a peptide or fragment or variant thereof by determining an immune response to the peptide. SEQ ID NO: 1 represents the full length of BP0205. The BP0205 sequence can also be found in NP_879093 (WP_010929682.1). Also disclosed herein is a vaccine comprising a peptide with 50%, 55%, 60%, 65%, 70%, 75%, 80%, 85%, 90%, 95%, 96%, 97%, 98%, or 99% identity to SEQ ID NO. 1.

It has been contemplated that BP0205 contains an adjuvant region near the amino terminus of the peptide. It should also be understood that an antigen differs from an adjuvant in that an antigen serves as the target ingredient or component of a vaccine to elicit an immune response, specifically by activating an adaptive immune response (such as, for example presentation to an MHC Class II cell and recognition by a CD4+ T cell). Whereas an adjuvant serves as a co-administered ingredient or component of a vaccine to increase the magnitude and durability of the immune response against the antigen (Moyer et al. “Beyond antigens and adjuvants: formulating future vaccines. 2016). Thus, an antigenic region of BP0205 in combination with at least one additional B. pertussis peptide elicits an immune response.

In some embodiments, the BP0205 peptide comprises at least one peptide fragment. In some embodiments, the BP0205 peptide comprises 1, 2, 3, 4, 5, or more peptide fragments. In some embodiments, the 1, 2, 3, 4, 5, or more peptide fragments are operably linked together by a peptide linker. As used herein, a “peptide linker” refers to a short sequence of amino acids used to separate 2 or more peptide fragments within a single peptide or protein. Peptide linkers can vary in length from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids in length. In some embodiments, the BP0205 peptide comprises SEQ ID NO: 2 and/or SEQ ID NO:3. In some embodiments, SEQ ID NO: 2 and SEQ ID NO: 3 are separated by 1, 2, 3, 4, 5, or more amino acids. In some embodiments, SEQ ID NO: 2 and SEQ ID NO: 3 are operably linked by a peptide linker.

As used herein, “operably linked refers to a juxtaposition of two or more components (as such peptide fragments), in which the components are arranged such that the two or more components maintain normal functions (such as, for example eliciting an immune response).

As used herein, an “antigen epitope”, “epitope”, or “antigenic determinant” refer to the part of an antigen, a molecular structure, or foreign particulate that can bind to a specific antibody or T-cell receptor. The presence of antigens or epitopes of antigens within a host can illicit an immune response. It should be understood that “antigen epitope”, “epitope”, or “antigenic determinant” can be used interchangeably.

In some embodiments, SEQ ID NO: 2, SEQ ID NO: 3, or SEQ ID NO: 4 can be individually used as an antigen epitope. In some embodiments, SEQ ID NO: 2, SEQ ID NO: 3, and SEQ ID NO: 4 can be arranged in any order or combination to achieve the desired immunogenic effect. Thus, in some embodiments, the pertussis vaccine comprises an antigen epitope comprising SEQ ID NO: 2; SEQ ID NO: 3; SEQ ID NO: 2 and SEQ ID NO: 3; SEQ ID NO. 3 and SEQ ID NO: 2; SEQ ID NO: 2 and SEQ ID NO: 4, SEQ ID NO: 4 and SEQ ID NO: 2; SEQ ID NO: 3 and SEQ ID NO: 4; SEQ ID NO: 4 and SEQ ID NO: 3; SEQ ID NO: 2, SEQ ID NO. 3, and SEQ ID NO: 4; SEQ ID NO. 2, SEQ ID NO: 4, and SEQ ID NO: 3; SEQ ID NO: 3, SEQ ID NO: 4, and SEQ ID NO: 2; SEQ ID NO: 3, SEQ ID NO: 2, and SEQ ID NO: 4; SEQ ID NO: 4, SEQ ID NO: 2, SEQ ID NO: 3; or SEQ ID NO: 4, SEQ ID NO: 3, and SEQ ID NO: 2.

In some embodiments, the BP0205 peptide comprises at least one peptide fragment of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or more amino acids. In some embodiments, said amino acids are contiguous. In some embodiments, said amino acids are noncontiguous. In some embodiments, said amino acids are near the carboxy terminus of BP0205. In some embodiments, said amino acids are near the amino terminus of BP0205. In some embodiments, 1, 2, 3, 4, 5, 6, 7, 8, 9, 10, 11, 12, 13, 14, 15, 16, 17, 18, 19, 20, or more amino acids are changed in the peptide fragment. In some embodiments, said amino acids are conserved. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140 or more amino acids are deleted from the BP0205 peptide. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140 or more amino acids are added to the BP0205 peptide.

In some embodiments, the pertussis vaccine comprises at least 50% sequence identity to SEQ ID NO. 1. In some embodiments, the pertussis vaccine comprises at least 55% sequence identity to SEQ ID NO: 1. In some embodiments, the pertussis vaccine comprises at least 60% sequence identity to SEQ ID NO: 1. In some embodiments, the pertussis vaccine comprises at least 65% sequence identity to SEQ ID NO: 1. In some embodiments, the pertussis vaccine comprises at least 70% sequence identity to SEQ ID NO: 1. In some embodiments, the pertussis vaccine comprises at least 75% sequence identity to SEQ ID NO: 1. In some embodiments, the pertussis vaccine comprises at least 80% sequence identity to SEQ ID NO: 1. In some embodiments, the pertussis vaccine comprises at least 85% sequence identity to SEQ ID NO: 1. In some embodiments, the pertussis vaccine comprises at least 90% sequence identity to SEQ ID NO: 1. In some embodiments, the pertussis vaccine comprises at least 95% sequence identity to SEQ ID NO: 1. In some embodiments, the pertussis vaccine comprises at least 96% sequence identity to SEQ ID NO. 1. In some embodiments, the pertussis vaccine comprises at least 97% sequence identity to SEQ ID NO: 1. In some embodiments, the pertussis vaccine comprises at least 98% sequence identity to SEQ ID NO: 1. In some embodiments, the pertussis vaccine comprises at least 99% sequence identity to SEQ ID NO: 1. In some embodiments, the pertussis vaccine comprises SEQ ID NO: 1.

In some embodiments, the pertussis vaccine comprises the BP020S peptide and 1, 2, 3, 4, 5, 6, 7, 8, 9, 10, 11, 12, 13, 14, 15, 16, 17, 18, 19, 20, or more peptides from a B. pertussis bacterium. In some embodiments, the 1, 2, 3, 4, 5, 6, 7, 8, 9, 10, 11, 12, 13, 14, 15, 16, 17, 18, 19, 20, or more peptides originates from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303 proteins, or combinations thereof, from Table 2 or from at least one biological pathway selected from cell adhesion and motility, cell cycle, cell signaling, cell structure, cell transport, gene regulation, metabolism, oxidation-reduction, protein regulation, or stress responses.

In some embodiments, the protein selected from cell adhesion and motility pathways comprise attaching and effacing protein, fimbrial protein, flagellar biosynthesis protein (FlhF), or combinations thereof.

In some embodiments, the protein selected from cell cycle pathways comprise beta-lactamase domain-containing protein, cell division coordinator (CpoB), cell division protein (ZapD), peptidyl-prolyl cis-trans isomerase, peptidyl-prolyl cis-trans isomerase (Cbf2), phospho-N-acetylmuramoyl-pentapeptide-transferase, tol-Pal system protein (TolB), tol-Pal system protein (TolQ), or combinations thereof.

In some embodiments, the protein selected from cell signaling pathways comprise dermonecrotic toxin, cyclic di-GMP phosphodiesterase response regulator (RpfG), enterobactin outer-membrane receptor, or combinations thereof.

In some embodiments, the protein selected from cell structure pathways comprise metal-binding protein, BrkB, D-alanine-D-alanine ligase, inner membrane protein (ybhN), inner membrane protein (YiaH), lipoprotein, lipoteichoic acid synthase 2, outer membrane protein assembly factor (BamA), P.93, penicillin-binding protein 1A, pertussis toxin liberation protein F, membrane protein, DMT superfamily permease, spermidine synthase with an N-terminal membrane domain, lipoate-protein ligase A, HflK, assembly protein, inner membrane protein, HI_0719, soluble lytic murein transglycosylase, virulence-associated outer membrane protein Vir-90, or combinations thereof.

In some embodiments, the protein selected from cell transport pathways comprise Xaa-Pro aminopeptidase, 2-aminoethylphosphonate ABC transporter substrate-binding protein, aerobactin synthase (IucC), antiseptic resistance protein, arabinose efflux permease, BFD-like [2Fe-2S] binding domain, bicarbonate transport ATP-binding protein (CmpD), bicyclomycin/multidrug efflux system, biotin transporter, BrkA autotransporter, ComEC family competence protein, D-methionine-binding lipoprotein (metQ), drug efflux system protein (MdtG), efflux pump membrane transporter, extracytoplasmic solute receptor protein, extracytoplasmic solute receptor protein (yiaO)), ferrichrome receptor (FcuA), filamentous hemagglutinin, heme-repressible hemoglobin-binding protein, hemin import ATP-binding protein (HmuV), inner membrane metabolite transport protein (yhjE), inner membrane protein (yhjX), inner membrane transport protein (ydhP), iron-sulfur cluster carrier protein, leucine ABC transporter subunit substrate-binding protein (LivK), leucine-, isoleucine-, valine-, threonine-, and alanine-binding protein, LIV-I protein F, LIV-I protein H, major facilitator superfamily proteins, manganese transport system membrane protein (mntB), methyl viologen resistance protein (SmvA), multidrug export ATP-binding/permease protein (SAV1866), multidrug resistance protein B, multiple resistance and pH homeostasis protein A, multiple resistance and pH homeostasis protein D, multiple resistance and pH homeostasis protein E, Neu5Ac permease, nickel/cobalt efflux system, BP0840, preprotein translocase subunit (SecD), ABC transporter ATP-binding protein (HI_0664), amino-acid metabolite efflux pump, chorismate pyruvate-lyase, membrane transporter protein, protein translocase subunit (SecD), purine efflux pump (PbuE), putrescine transport system permease protein (PotH), RTX-I toxin determinant B, sec-independent protein translocase protein (TatC), short-chain fatty acids transporter, sn-glycerol-3-phosphate transport system permease protein (ugpA), spermidine/putrescine transport system permease protein (PotB), TonB family C-terminal domain, TpsB transporter, transport permease protein, TRAP-type C4-dicarboxylate transport system, small permease component, TRAP-type uncharacterized transport system, fused permease components, tripartite tricarboxylate transporter (TctA), or combinations thereof.

In some embodiments, the protein selected from gene regulation pathways comprise type IV secretion system protein virB11, 30S ribosomal protein S10, 50S ribosomal protein L10, 50S ribosomal protein L14, 50S ribosomal protein L25, 50S ribosomal protein L31 type B, ATP-dependent DNA helicase Rep, ATP-dependent RNA helicase (HrpA), bacterial DNA-binding protein, BpH3, cyn operon transcriptional activator, DNA polymerase III subunit gamma/tau, DNA protection during starvation protein 2, DNA repair protein (RecN), galactose-binding protein regulator, Gcv operon activator, HTH-type transcriptional regulatory protein (gabR), leucine-responsive regulatory protein, LigA, polyribonucleotide nucleotidyltransferase, protein (ApaG), ribonuclease (TTHA0252), ribosomal RNA small subunit methyltransferase B, ribosome-binding factor A, ribosome-recycling factor, single-stranded DNA-binding protein, transcriptional regulator, y4mF family, transcription-repair-coupling factor, or combinations thereof.

In some embodiments, the protein selected from metabolic pathways comprise ACR, COG1565, 1,4-alpha-glucan branching enzyme (GlgB), 1,4-dihydroxy-2-naphthoyl-CoA synthase, 2-(hydroxymethyl) glutarate dehydrogenase, 2,3,4,5-tetrahydropyridine-2,6-dicarboxylate N-acetyltransferase, 3-(3-hydroxy-phenyl) propionate/3-hydroxycinnamic acid hydroxylase, 3-hydroxyacyl-CoA dehydrogenase, 3-oxoadipate enol-lactonase 2, 3-oxosteroid 1-dehydrogenase, 4-aminobutyrate aminotransferase PuuE, 4-hydroxy-3-methylbut-2-en-1-yl diphosphate synthase (flavodoxin), 5-formyltetrahydrofolate cyclo-ligase, 5-methyltetrahydropteroyltriglutamate—homocysteine methyltransferase, 6-aminohexanoate-cyclic-dimer hydrolase acetaldehyde dehydrogenase 2, acetolactate synthase isozyme 3 large subunit, acetone carboxylase alpha subunit, acetone carboxylase beta subunit, acetyl-CoA acetyltransferase, acetyltransferase component of pyruvate dehydrogenase complex, acyl carrier protein, acyl-[acyl-carrier-protein]-UDP-N-acetylglucosamine O-acyltransferase, acylase (ACY 1), acyl-CoA dehydrogenase (AidB), short-chain specific acyl-CoA dehydrogenase, adenosylhomocysteinase, aldehyde dehydrogenase, thermostable aliphatic sulfonates transport permease protein (ssuC), amidophosphoribosyltransferase, aminotransferase, arginine transport ATP-binding protein (ArtM), argininosuccinate lyase, aspartate 1-decarboxylase, aspartoacylase, aspartokinase, ATP synthase subunit beta, ATP-dependent dethiobiotin synthetase (BioD), beta-ketoacyl-acyl-carrier-protein synthase I, biotin carboxyl carrier protein of acetyl-CoA carboxylase, biotin carboxylase, carbamoyl-phosphate synthase large chain, carbamoyl-phosphate synthase small chain, cytochrome c, cytochrome c oxidase subunit 2, D-amino acid dehydrogenase, dihydrolipoyl dehydrogenase, dihydrolipoyllysine-residue succinyltransferase component of 2-oxoglutarate dehydrogenase complex, dihydropteroate synthase, D-malate degradation protein R, D-malate dehydrogenase (decarboxylating), exported protein, ferredoxin-dependent glutamate synthase 1, Flp pilus assembly protein (TadD), formate-dependent phosphoribosylglycinamide formyltransferase, formyl-coenzyme A transferase, glutamate dehydrogenase, glutamine—fructose-6-phosphate aminotransferase, glyceraldehyde-3-phosphate dehydrogenase, glycine dehydrogenase, guanidinobutyrase, imidazoleglycerol-phosphate dehydratase, iron-sulfur cluster assembly scaffold protein (IscU), L-aspartate dehydrogenase, long-chain-fatty-acid—CoA ligase, malate dehydrogenase, maltose alpha-D-glucosyltransferase, MmgE/PrpD family, N5-carboxyaminoimidazole ribonucleotide synthase, NAD⁺ binding domain of 6-phosphogluconate dehydrogenase, nicotinamidase/pyrazinamidase, nucleoside recognition, Oxoglutarate dehydrogenase, phosphoserine aminotransferase, hydrolase of the alpha/beta-hydrolase fold, protoheme IX biogenesis protein, soluble aldose sugar dehydrogenase (yliI), sporulation inhibitor (kipI), stringent starvation protein A, succinate dehydrogenase flavoprotein subunit, succinate-semialdehyde dehydrogenase [NADP(+)] (GabD), thiamine biosynthesis protein (HI_0357), thiosulfate sulfurtransferase, threonine synthase, thymidylate synthase, UDP-glucose 6-dehydrogenase, Vi polysaccharide biosynthesis protein (TviD), xanthine dehydrogenase accessory protein (XdhC), or combinations thereof.

In some embodiments, the protein selected from oxidation-reduction pathways comprise 3-alpha-(Or 20-beta)-hydroxysteroid dehydrogenase, 3-ketosteroid-9-alpha-hydroxylase reductase subunit, 3-oxoacyl-[acyl-carrier-protein] reductase (FabG), acetoin: 2,6-dichlorophenolindophenol oxidoreductase subunit beta, alkyl hydroperoxide reductase C, benzaldehyde dehydrogenase [NAD(+)], benzene 1,2-dioxygenase subunit alpha, catalase, dTDP-4-dehydrorhamnose reductase, gamma-glutamylputrescine oxidoreductase, gluconate 2-dehydrogenase cytochrome c subunit, glutathione hydrolase proenzyme, glutathione import ATP-binding protein (GsiA), glutathione transport system permease protein (gsiD), glutathione-binding protein (gsiB), glyoxylate/hydroxypyruvate reductase A, glyoxylate/hydroxypyruvate reductase B, heme A synthase, L-2-hydroxyglutarate oxidase (LhgO), methylmalonate-semialdehyde dehydrogenase, Mg-chelatase subunit child, muconolactone delta-isomerase, NAD(P)H-hydrate epimerase, NADH-quinone oxidoreductase, NADH-quinone oxidoreductase subunit L, NADH-quinone oxidoreductase subunit M, NADH-quinone oxidoreductase subunit N, aldo-keto reductase, NAD(P)H quinone oxidoreductase, PIG3, quinone oxidoreductase 1, rhodocoxin reductase, sarcosine oxidase subunit beta, sulfite reductase, thiol:disulfide interchange protein (DsbD), thioredoxin reductase, ubiquinol oxidase subunit 1, or combinations thereof.

In some embodiments, the protein selected from protein regulation pathways comprise apolipoprotein N-acyltransferase, ATP-dependent Clp protease ATP-binding subunit (ClpX), capsule polysaccharide biosynthesis protein, carboxypeptidase G2, chaperonin GroEL, chorismate synthase, co-chaperonin GroES, dihydroxy-acid dehydratase, FtsH protease regulator (HfIK), small chain glutamate synthase, glutamine synthetase, lon protease, membrane dipeptidase M19, membrane protein insertase (YidC), methionine-tRNA ligase, molybdopterin molybdenumtransferase, oligopeptidase A, periplasmic serine endoprotease DegP-like, C-terminal PDZ domain protease, HtpX, protein-L-isoaspartate O-methyltransferase, glutamate—cysteine ligase 2, tRNA pseudouridine synthase A, tryptophan-tRNA ligase, tyrosine-tRNA ligase, or combinations thereof.

In some embodiments, the protein selected from stress response pathways comprise Rv1996/MT2052, SAV1710, or combinations thereof.

It should be noted that the pertussis vaccine of the present disclosure is also an immunogenic composition comprising a BP0205 antigen, or variants thereof, in combination with at least one peptide from a B. pertussis bacterium. It should be further understood that vaccine and immunogenic composition can be used interchangeably. Thus, the vaccine further comprises a pharmaceutically acceptable carrier, and one or more adjuvants, preservatives, or stabilizers, or any combinations thereof. Herein, the term “pharmaceutically acceptable” means that the carrier or excipient, at the dosages and concentrations employed, will not cause unwanted or harmful effects in the subjects to which they are administered. Such pharmaceutically acceptable carriers and excipients are well known in the art (Remington. The Science and Practice of Pharmacy, Mack Publishing Company 1990; Frokjaer, S. & Hovgaard, L. Pharmaceutical Formulation Development of Peptides and Proteins, 2000; Handbook of Pharmaceutical Excipients, Pharmaceutical Press 2000). The compositions preferably are formulated and administered as a sterile solution. Sterile Solutions are prepared by sterile filtration or by other methods known per se in the art. The solutions can then be lyophilized or filled into pharmaceutical dosage containers. The pH of the solution generally is in the range of pH 3.0 to 9.5, e.g., pH 5.0 to 7.5. The components of the composition typically are in a solution having a suitable pharmaceutically acceptable buffer, and the solution may also contain a salt. In some embodiments, detergent is present in the vaccine. In some embodiments, the vaccine may be formulated into an injectable preparation. These vaccine formulations contain effective amounts of the antigen and/or peptide components, are either sterile liquid solutions, liquid suspensions, or lyophilized versions. The vaccine formulations can also comprise a non-limiting quantity or combinations of adjuvants, stabilizers, preservatives, and/or excipients.

Further examples of suitable formulations for the storage and/or pharmaceutical administration of pertussis vaccines are known (“Vaccines” 5^{th} edition. S. Plotkin, et al). Examples of suitable diluents are PBS or saline. Herein, the pertussis vaccine can have at least one preservative present, including, but not limited to phenoxyethanol, thimerosal, or parabens. If a preservative is present, it is preferably present at low levels.

In some embodiments, the pertussis vaccine comprises at least one adjuvant. Adjuvants are known in the art to further increase the immune response to an applied antigenic determinant (for a review on adjuvants, see, e.g., Montomoli, 2011, Expert Rev. Vaccines 10:1053-1061). Examples of suitable adjuvants include, but are not limited to aluminum salts such as aluminum hydroxide and/or aluminum phosphate; oil-emulsion compositions (or oil-in-water compositions), including squalene water emulsions, such as MF59 (see, e.g., WO 90/14837): saponin formulations, such as, for example, QS21 and Immunostimulating Complexes (ISCOMS) (see, e.g., U.S. Pat. No. 5,057,540; WO 90/03184, WO 96/11711, WO 2004/004762, WO 2005/002620); Toll-like receptor (TLR) agonists, e.g., a TLR7 agonist (see, e.g., WO 2012/117377, page 15-18, for examples), e.g., in combination with an aluminum salt, e.g., aluminum hydroxide to which the TLR agonist may be adsorbed; bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as 35 E. coli heat labile enterotoxin LT, cholera toxin CT, and the like.

Methods Preventing an Infection and/or Improving an Immune Response

In one aspect, disclosed herein is a method of preventing an infection from a B. pertussis bacterium in a subject, the method comprising administering to the subject a pharmaceutically effective amount of the pertussis vaccine of any preceding aspect.

In one aspect, disclosed herein is a method of improving an immune response against a B. pertussis bacterium in a subject, the method comprising administering to the subject a pharmaceutically effective amount of the pertussis vaccine of any preceding aspect.

In one aspect, disclosed herein are methods of preventing an infection from a B. pertussis bacterium in a subject, wherein the subject is administered a pharmaceutically effective amount of a pertussis vaccine to enhance an immune response.

In some embodiments, the infection from the B. pertussis bacterium causes whooping cough in the subject. It should be noted that whooping cough, as a result of B. pertussis infections, is a highly contagious and highly communicable disease affecting the respiratory system. Individuals impacted by this infection display severe hacking coughs followed by a high-pitched intake of breath that sounds like a “whoop”. Although this disease was once considered to be a childhood disease, it is now known that whooping cough can cause serious illness in individuals of all ages, including infants, toddlers, children, adolescents, young adults, adults, elderly, and pregnant women. It is also appreciated in the art that the best way to prevent B. pertussis and thus whooping cough is through vaccine administration (“Whooping Cough Vaccination” August 2022. www.cdc.gov/pertussis/vaccines).

In some embodiments, the immune response comprises presenting the antigen to an MHC Class II cell, recognition of the antigen by a CD4+ T cell, or combination thereof. In some embodiments, the immune response comprises a cellular (T cell) and/or a humoral (antibody) response. In some embodiments, the immune response is against a BP0205 antigen and at least one protein involved in at least one biological pathway within the B. pertussis bacterium. In some embodiments, the at least one pathway selected from cell adhesion and motility, cell cycle, cell signaling, cell structure, cell transport, gene regulation, metabolism, oxidation-reduction, protein regulation, or stress responses.

In some embodiments, the method further comprises administering the pertussis vaccine through intranasal, intramuscular, intraperitoneal, or subcutaneous routes of injection. In some embodiments, the method comprises administering an acellular pertussis vaccine comprising a BP0205 antigen, or variants thereof, in combination with at least one peptide from a B. pertussis bacterium, wherein the BP0205 peptide and the at least one peptide are to be presented to an MHC Class II cell and recognized by a CD4+ T cell. In some embodiments, the method comprises administering a pertussis vaccine (such as, for example an aP vaccine) comprising the BP0205 antigen and 1, 2, 3, 4, 5, 6, 7, 8, 9, 10, 11, 12, 13, 14, 15, 16, 17, 18, 19, 20, or more peptides from a B. pertussis bacterium. In some embodiments, the method comprises 1, 2, 3, 4, 5, 6, 7, 8, 9, 10, 11, 12, 13, 14, 15, 16, 17, 18, 19, 20, or more peptides originating from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303 proteins, or combinations thereof, from Table 2 or from at least one biological pathway selected from cell adhesion and motility, cell cycle, cell signaling, cell structure, cell transport, gene regulation, metabolism, oxidation-reduction, protein regulation, or stress responses.

The pertussis vaccine may be administered in such amounts, time, and route deemed necessary in order to achieve the desired result. The exact amount of the pertussis vaccine will vary from subject to subject, depending on the species, age, and general condition of the subject, the severity of the B. pertussis infection, the particular vaccine formulation, its mode of administration, its mode of activity, and the like. The pertussis vaccine is preferably formulated in dosage unit form for ease of administration and uniformity of dosage. The specific therapeutically effective dose level for any particular subject will depend upon a variety of factors including the infection being treated and the severity of the infection, the activity of the pertussis vaccine employed; the specific pertussis vaccine employed; the age, body weight, general health, sex and diet of the patient; the time of administration, route of administration, and rate of excretion of the specific pertussis vaccine employed; the duration of the treatment; drugs used in combination or coincidental with the specific pertussis vaccine employed; and like factors well known in the medical arts.

The pertussis vaccine may be administered by any route. In some embodiments, the pertussis vaccine is administered via a variety of routes, including intravenous, intramuscular, intra-arterial, intramedullary, intrathecal, subcutaneous, intraventricular, transdermal, interdermal, rectal, intravaginal, intraperitoneal, mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the pertussis vaccine (e.g., its stability in the environment of the subject's body), the condition of the subject (e.g., whether the subject is able to tolerate administration), etc. In one embodiment the vaccine is administered by intramuscular injection.

The exact amount of pertussis vaccine required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

The pertussis vaccine is also suitably used as a booster vaccine for populations that have been previously vaccinated by other vaccines, be those of whole-cell pertussis vaccine (wP) or aP vaccines of different composition or combination vaccines comprising wP or aP of different composition than the vaccines of the disclosure. Such boosters may, for instance, be used for vaccination of adults or elderly that have not been vaccinated against B. pertussis for more than a decade. It could be useful to repeat such booster vaccinations about once every five, ten, or fifteen years. In certain embodiments, the aP is suitable for administration to an infant, a child, an adolescent, an adult, an elderly, or a pregnant woman.

A dose of a vaccine is the amount that is administered in a single administration to a subject. A subject may suitably be an animal or a human, and in some embodiments the subject is a human. In some embodiments, the subject is a mammal. Vaccines disclosed here are suitably administered at least one time to the same individual to obtain a desirable effect against infection. Vaccines disclosed herein are suitably administer more than one time to the same individual with sufficient time interval to obtain a boosting effect in said individual (such as, for example a time interval of at least four weeks to 6 months to one year to several years and up to two decades). A non-limiting example of pertussis vaccine administration includes two or three or more times with at least 4 weeks interval, for instance, a one or two month time interval between each administration. One non limiting example is administration according to the EPI schedule, at 6 weeks, 10 weeks and 14 weeks of age. Another regimen would be at 2 months, 4 months, 6 months of age. In some embodiments, a booster vaccination comprising the pertussis vaccine is given 10-20 years later (such as, for example during adolescence). In some embodiments, the pertussis vaccine administration comprises two or three times in the first year of life, a further boost is administered the second year of life, and a further booster is administered at four to five years of age, after which an adolescent boost is administered at approximately twelve years of age.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Unbiased Biochemical Identification of MHC Class II Presented Immunogenic Epitopes from Bordetella pertussis

### Methods

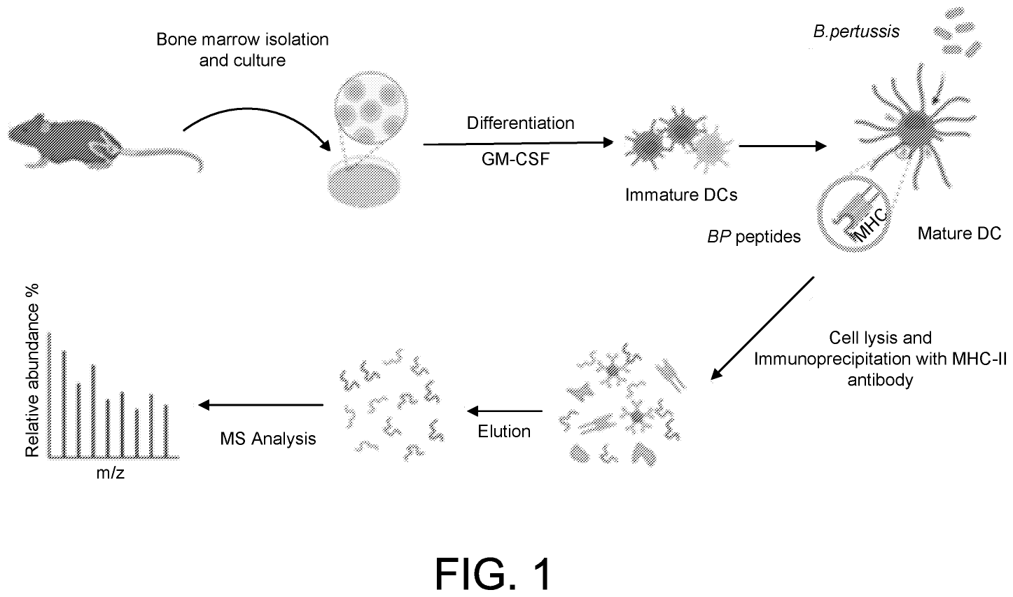

Immunoprecipitation of MHC-II epitopes. Bone marrow was isolated from WT C57BL/6J mice and cells resuspended in dendritic cell (DC) differentiation media composed of RPMI1640 supplemented with 10% FBS, 10 μg/ml gentamicin, ß-mercaptoethanol and 40 ng/μl GM-CSF. 10×10⁶ cells were then seeded in 10 cm non-tissue culture dish and differentiated for 7 days. Bone marrow derived dendritic cells (BMDDCs) were then transferred to tissue culture treated plates and left to settle for 24 hrs; meanwhile, Bordetella pertussis was cultured overnight. The next day, either live H762 or heat killed BP536 were co-cultured with BMDCs at an MOI of 100 for 24 hours. BMDCs were then harvested by scraping and were frozen until ready for immunoprecipitation (IP). To do MHCII IP, frozen cell pellets were quick thawed and lysed in cold IP lysis buffer composed of 150 mM NaCl, 4 mM MgCl2, 0.25 mM CaCl2, 20 mM Tris pH 8, 1% CHAPS, 6 μg/ml DNasel from bovine pancreas (Sigma Aldrich), protease inhibitor cocktail (Sigma Aldrich) and 1 mM PMSF (RPI) at 5×10⁷ cells/ml lysis buffer/sample. Clarified lysates were subjected to immunoprecipitation overnight at 4° C. with gentle rotation. Immunoprecipitation was performed with Protein G sepharose beads (GE Healthcare) coupled to αMHC-II antibody (BioXcell, clone M5/114). Each sample contained around 2 mg bound antibody Beads were then washed thrice with sterile cold PBS followed by sterile cold ultrapure water. Finally, samples were freeze dried and stored at −80 until peptide isolation and detection. Acid elution and detection of the peptides using liquid chromatography-tandem mass spectrometry (LC-MS/MS) was done according to a protocol providing a comprehensive list of MHCII displayed peptide epitopes. FIG. 1 shows the experiment workflow.

Analysis of and refining the MHCII epitope library. To refine the list of antigens, the list of epitopes in silico for their MHC-II binding affinity were examined using the Immune Epitope Database (IEDB). This online database contains an extensive repository of experimentally validated immune epitopes. The whole peptide library was interrogated using IEDB for binding to mouse and human MHCII alleles and high affinity binders (defined as having adjusted percentile rank of ≤10) were detected.

Splenocyte stimulation and cytokine ELISA assays. The ability of the identified peptides to activate T cells from immunized or convalescent mice was tested. Mice challenged with 5×10⁵ CFUs bacteria (either BP536 or H762) were used at >35 days post-challenge. Mice immunized intramuscularly with 1×10⁸ CFUs of heat killed bacteria with 2 doses 1 month apart and analyzed at least 2 weeks post-boost, were used as wPV immunized mice. Following dissociation and red blood cell lysis using ACK buffer, a single-cell suspension was plated at 2.5×10⁶ cells/well of complete T cell medium (RPMI, 10% FBS, 10 μg/ml gentamicin, 5×10⁻⁵ M ß-mercaptoethanol) and stimulated with 1 μg/ml of selected peptides or with medium alone as a negative control. The supernatant was collected on day 3 post stimulation. The production of IFNγ was quantified by a sandwich ELISA (R&D Cat. DY485-05) according to the manufacturer's instructions. Plates were read at A450 on a SpectraMax i3X® plate reader and concentration calculated based on the standard curve.

³H Thymidine incorporation. The ability of the identified peptides to induce proliferation of T cells isolated from immunized or convalescent mice was tested. Following dissociation and red blood cell lysis using ACK buffer, a single-cell suspension was plated at 4×10⁵ cells/well of complete T cell medium (RPMI, 10% FBS, 10 μg/ml gentamicin, 5×10⁻⁵ M ß-mercaptoethanol) and stimulated with 10 μg/ml of selected peptides or with medium alone as a negative control for 3 days. Cells were then pulsed with ³H-thymidine-media (RPMI 1640 with 1.25% HEPES buffer and 2 μCi/ml ³H-thymidine) for 18 h. Cells were then harvested with the FilterMate Harvester (PerkinElmer, Shelton, CT) onto Unifilter plates and stored overnight at room temperature to dry. 30 μl of Microscint20 (PerkinElmer) was added to each well and plates were sealed. 3H-thymidine incorporation was read using a TopCount NXT machine (PerkinElmer).

PBMC collection. Peripheral blood obtained from consented volunteers is mixed 1:1 with 0.9% saline and loaded on Ficoll-paque (at 25° C.) and centrifuged in a swing-bucket centrifuge (Eppendorf 5810R) 30 min at 1500 rpm, 25° C. using no brake. PBMCs at the interface are collected and washed twice with RPMI 1640+2% HI-FBS, Cells are pelleted for 5 min at 1500 rpm, 4° C., the supernatant is aspirated and the cells are resuspended in 90% FBS+10% DMSO, aliquoting 1.0×10\hat{ }6 cells per cryovial. The harvested PBMCs are frozen at −80° C. and stored for downstream applications.

Activation-induced marker (AIM) assay. Frozen PBMC samples are resuspended in T cell media (RPMI 1640+10% AB sera+10 μg/ml Gentamicin+0.2% Beta mercaptoethanol. Cells are pelleted for 5 min at 1500 rpm, 4° C., and resuspended in T cell media for counting. 100 μL of PBMCs per well are added to a sterile U bottom 96 well plate (Falcon Ref 353077) (2×10⁶/well). 100 μL of the following prepared stimuli (1 μg/ml of selected peptide, phytohemagglutinin (PHA) for positive control, and DMSO was added for a negative control) and mixed by pipetting. Samples are incubated overnight ˜18 hours at 37° C.+5% CO₂. Next day, cells are pelleted and washed 2× with cold sterile 1×PBS. Cells are resuspended in Live/Dead stain (Zombie Nir 1:10,000 dilution) and incubated for 30 min in the dark at 4° C. Cells are washed 2× in PBS, and samples are blocked with 10% FBS PBS for 5 min at 4° C. Following blocking, antibodies are added (Table 1) and FMOs are prepared using pooled cells for gating controls. Samples incubate for 15 min at 4° C. Following labeling, samples are washed as highlighted above cells are resuspended in 1% FBS PBS and transferred into FACs tubes. Cells are analyzed using a Cytek Aurora spectral flow cytometer. Compensation beads (eBioscience Ultra comp beads) are labeled for compensation. Data are analyzed using FlowJo and GraphPad Prism is used for figure generation.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### TABLES

TABLE 1
Human AIM assay antibody panel
Compensation controls

| Marker and | | |
|---|---|---|
| Dilution | Host | Conjugation |
| Unstained | | |
| control | | |
| OX40 (1/1000) | Mouse | PE-Cy7 |
| CCR7 (1/250) | Mouse | PerCP/Cy5.5 |
| CD19 (1/1000) | Mouse | V500 |
| CD4 (1/1000) | Mouse | APCef780 |
| CD3 (1/1000) | Mouse | AF700 |
| PD-L1 (1/1000) | Mouse | PE |
| CD45A (1/1000) | Mouse | ef450 |
| CD25 (1/250) | Mouse | FITC |
| CD69 (1/1000) | Mouse | BV605 |
| CD137 (1/1000) | Mouse | APC |

TABLE 2
B. pertussis Proteins of Interest

| Protein Name | Accession Number |
|---|---|
| 1,4-alpha-glucan branching enzyme | A0A381A1T1 |
| GlgB | |
| 1,4-Dihydroxy-2-naphthoyl-CoA | A0A0E8EKU6 |
| synthase | |
| 2-(Hydroxymethyl)glutarate | A0A0E8CG08 |
| dehydrogenase | |
| 2,3,4,5-tetrahydropyridine-2,6- | Q79A64 |
| dicarboxylate N-acetyltransferase | |
| 2-aminoethylphosphonate ABC | A0A0E8DKJ0 |
| transporter substrate-binding protein | |
| 3-(3-hydroxy-phenyl)propionate/3- | A0A381A7H5 |
| hydroxycinnamic acid hydroxylase | |
| 30S ribosomal protein S10 | A0A0E7U3C9 |
| 3-alpha-(Or 20-beta)-hydroxysteroid | A0A0E7USS6 |
| dehydrogenase | |
| 3-hydroxyacyl-CoA dehydrogenase | A0A381A8C8 |
| 3-ketosteroid-9-alpha-hydroxylase | A0A381A4X4 |
| reductase subunit | |
| 3-oxoacyl-[acyl-carrier-protein] | A0A381A5E2 |
| reductase FabG | |
| 3-oxoadipate enol-lactonase 2 | A0A0E7USV0 |
| 3-oxosteroid 1-dehydrogenase | A0A0E8DK41 |
| 4-aminobutyrate aminotransferase | A0A381A3K5 |
| PuuE | |
| 4-hydroxy-3-methylbut-2-en-1-yl | A0A381A415 |
| diphosphate synthase (flavodoxin) | |
| 50S ribosomal protein L10 | A0A0E8ECD2 |
| 50S ribosomal protein L14 | A0A0E7U3C1 |
| 50S ribosomal protein L25 | A0A0E7UTR5 |
| 50S ribosomal protein L31 type B | A0A0E8BKF9 |
| 5-formyltetrahydrofolate cyclo- | A0A381A630 |
| ligase | |
| 5- | A0A0E8FAG3 |
| methyltetrahydropteroyltriglutamate-- | |
| homocysteine methyltransferase | |
| 6-aminohexanoate-cyclic-dimer | A0A381A3Z4 |
| hydrolase | |
| Acetaldehyde dehydrogenase 2 | A0A381A4D2 |
| Acetoin:2,6- | A0A0E8DTT6 |
| dichlorophenolindophenol | |
| oxidoreductase subunit beta | |
| Acetolactate synthase isozyme 3 | A0A381A381 |
| large subunit | |
| Acetone carboxylase alpha subunit | A0A381A162 |
| Acetone carboxylase beta subunit | A0A381A3J8 |
| Acetyl-CoA acetyltransferase | A0A380ZYJ3 |
| Acetyltransferase component of | A0A381A112 |
| pyruvate dehydrogenase complex | |
| Acyl carrier protein | A0A0E8BLR4 |
| Acyl-[acyl-carrier-protein]--UDP-N- | A0A0E7UDT3 |
| acetylglucosamine O-acyltransferase | |
| Acylase ACY 1 | A0A0E8DRY8 |
| Acyl-CoA dehydrogenase AidB | A0A381A2T5 |
| Acyl-CoA dehydrogenase, short- | A0A0ESEKS7 |
| chain specific | |
| Adenosylhomocysteinase | A0A0E7UQY7 |
| Aerobactin synthase lucC | A0A0E8D783 |
| Aldehyde dehydrogenase, | A0A380ZYK6 |
| thermostable | |
| Aliphatic sulfonates transport | A0A0E8DVW5 |
| permease protein ssuC | |
| Alkyl hydroperoxide reductase C | A0A0E8C4Q8 |
| Amidophosphoribosyltransferase | A0A381ASV4 |
| Aminotransferase | A0A0E8F5G8 |
| Antiseptic resistance protein | A0A0E8DNH0 |
| Apolipoprotein N-acyltransferase | A0A381A4M2 |
| Arabinose efflux permease | A0A381A708 |
| Arginine transport ATP-binding | A0A0E8E5D3 |
| protein ArtM | |
| Argininosuccinate lyase | A0A0E7UDV4 |
| Aspartate 1-decarboxylase | A0A0E8BNA5 |
| Aspartoacylase | A0A0E7URG8 |
| Aspartokinase | A0A0E8EY18 |
| ATP synthase subunit beta | A0A0E8DRP5 |
| ATP-dependent Clp protease ATP- | A0A0E8C8B7 |
| binding subunit ClpX | |
| ATP-dependent dethiobiotin | A0A381A439 |
| synthetase BioD | |
| ATP-dependent DNA helicase Rep | A0A0E8EV11 |
| ATP-dependent RNA helicase HrpA | A0A0E8C5Y5 |
| Attaching and effacing protein | A0A381A0F0 |
| Bacterial DNA-binding protein | P94344 |
| Benzaldehyde dehydrogenase | A0A381A9D1 |
| [NAD(+)] | |
| Benzene 1,2-dioxygenase subunit | A0A0E8DHS2 |
| alpha | |
| Beta-ketoacyl-acyl-carrier-protein | A0A0E8EKG6 |
| synthase I | |
| Beta-lactamase domain-containing | A0A381A328 |
| protein | |
| BFD-like [2Fe-2S] binding domain | A0A0E8CFG5 |
| Bicarbonate transport ATP-binding | A0A0E7UVQ8 |
| protein CmpD | |
| Bicyclomycin/multidrug efflux | A0A0E8EV38 |
| system | |
| Biotin carboxyl carrier protein of | A0A0E7U211 |
| acetyl-CoA carboxylase | |
| Biotin carboxylase | A0A0E8C3J7 |
| Biotin transporter | A0A380ZZN8 |
| BpH3 | O07507 |
| BrkA autotransporter | A0A380ZYW1 |
| BrkB | Q45339 |
| Capsule polysaccharide biosynthesis | A0A381A3V2 |
| protein | |
| Carbamoyl-phosphate synthase large | A0A0E8CHJ3 |
| chain | |
| Carbamoyl-phosphate synthase | A0A0E7URX0 |
| small chain | |
| Carboxypeptidase G2 | A0A0E8CEL3 |
| Catalase | A0A0E8DM81 |
| Cell division coordinator CpoB | A0A381A0N8 |
| Cell division protein ZapD | A0A0E8ERX6 |
| Chaperonin GroEL | A0A0E8E5E7 |
| Chorismate synthase | A0A381A610 |
| Co-chaperonin GroES | A0A0E8CGD4 |
| ComEC family competence protein | A0A380ZZM2 |
| Cyclic di-GMP phosphodiesterase | A0A381A081 |
| response regulator RpfG | |
| Cyn operon transcriptional activator | A0A380ZXG6 |
| Cytochrome c | A0A381A4U1 |
| Cytochrome c oxidase subunit 2 | A0A0E8DCV0 |
| D-alanine--D-alanine ligase | A0A0E7UWL5 |
| D-amino acid dehydrogenase | A0A381A6C1 |
| Dermonecrotic toxin | Q45336 |
| Dihydrolipoyl dehydrogenase | A0A0E7UBR5 |
| Dihydrolipoyllysine-residue | A0A381A121 |
| succinyltransferase component of 2- | |
| oxoglutarate dehydrogenase | |
| complex | |
| Dihydropteroate synthase | A0A0E8CBT0 |
| Dihydroxy-acid dehydratase | A0A0E7UJL5 |
| D-malate degradation protein R | A0A0E7UPB3 |
| D-malate dehydrogenase | A0A381A1X1 |
| (decarboxylating) | |
| D-methionine-binding lipoprotein | A0A0E8D2W2 |
| metQ | |
| DNA polymerase III subunit | A0A381A4X1 |
| gamma/tau | |
| DNA protection during starvation | A0A0E7U4J2 |
| protein 2 | |
| DNA repair protein RecN | A0A381A5J3 |
| Domain of uncharacterized function | A0A0E8CQ90 |
| (DUF1983) | |
| Drug efflux system protein MdtG | A0A380ZY15 |
| dTDP-4-dehydrorhamnose reductase | A0A381A2C0 |
| DUF2591 domain-containing protein | A0A0E8BUY4 |
| Ectoine hydroxylase | A0A380ZWE2 |
| Ectoine utilization protein EutE | A0A381A534 |
| Efflux pump membrane transporter | A0A380ZZZ6 |
| Enterobactin outer-membrane | A0A0E8DN24 |
| receptor | |
| Exported protein | A0A0E8D061 |
| Extracytoplasmic solute receptor | A0A0ESEFX9 |
| protein | |
| Extracytoplasmic solute receptor | A0A0E8D0N6 |
| protein | |
| Extracytoplasmic solute receptor | A0A0E8DFQ1 |
| protein yiaO | |
| Ferredoxin-dependent glutamate | A0A0E8DVE7 |
| synthase 1 | |
| Ferrichrome receptor FcuA | A0A380ZXJ6 |
| Filamentous hemagglutinin | A0A381A265 |
| Fimbrial protein | Q6JHT0 |
| Flagellar biosynthesis protein FlhF | A0A381A2L9 |
| Flp pilus assembly protein TadD, | A0A0E8DJC0 |
| contains TPR repeats | |
| Formate-dependent | A0A0E8EI71 |
| phosphoribosylglycinamide | |
| formyltransferase | |
| Formyl-coenzyme A transferase | A0A380ZZY2 |
| FtsH protease regulator HflK | A0A0E7V3E3 |
| Galactose-binding protein regulator | A0A381A3S7 |
| Gamma-glutamylputrescine | A0A381A1F0 |
| oxidoreductase | |
| Gcv operon activator | A0A381A5V1 |
| Gluconate 2-dehydrogenase | A0A381A2F8 |
| cytochrome c subunit | |
| Glutamate dehydrogenase | A0A0E8FKJ2 |
| Glutamate synthase [NADPH] small | A0A0E8CM00 |
| chain | |
| Glutamine synthetase | A0A0E7UFW1 |
| Glutamine--fructose-6-phosphate | A0A0E8EEE3 |
| aminotransferase [isomerizing] | |
| Glutathione hydrolase proenzyme | A0A380ZZJ2 |
| Glutathione import ATP-binding | A0A0E8CT63 |
| protein GsiA | |
| Glutathione transport system | A0A0E8E463 |
| permease protein gsiD | |
| Glutathione-binding protein gsiB | A0A0E7V3I9 |
| Glyceraldehyde-3-phosphate | A0A0E8CJP6 |
| dehydrogenase | |
| Glycine dehydrogenase | A0A381A2L3 |
| (decarboxylating) | |
| Glyoxylate/hydroxypyruvate | A0A380ZYS2 |
| reductase A | |
| Glyoxylate/hydroxypyruvate | A0A0E7UNV2 |
| reductase B | |
| Guanidinobutyrase | A0A0E7UE97 |
| Heme A synthase | A0A0E8F783 |
| Heme-repressible hemoglobin- | A0A380ZY40 |
| binding protein | |
| Hemin import ATP-binding protein | A0A380ZY95 |
| HmuV | |
| HTH-type transcriptional regulatory | A0A381A3D6 |
| protein gabR | |
| Imidazoleglycerol-phosphate | A0A0E8CNA8 |
| dehydratase | |
| Inner membrane metabolite transport | A0A0E7UQP7 |
| protein yhjE | |
| Inner membrane protein ybhN | A0A381A0Z0 |
| Inner membrane protein yhjX | A0A381A5C4 |
| Inner membrane protein YiaH | A0A381A6S9 |
| Inner membrane transport protein | A0A380ZXZ2 |
| ydhP | |
| Iron-sulfur cluster assembly scaffold | A0A0E8D6I1 |
| protein IscU | |
| Iron-sulfur cluster carrier protein | A0A0E8ERV6 |
| L-2-hydroxyglutarate oxidase LhgO | A0A381A6P7 |
| L-aspartate dehydrogenase | A0A381A7D6 |
| Leucine ABC transporter subunit | A0A0E8DZ82 |
| substrate-binding protein LivK | |
| Leucine-, isoleucine-, valine-, | A0A0E8E0B9 |
| threonine-, and alanine-binding | |
| protein | |
| Leucine-responsive regulatory | A0A0E8FKZ4 |
| protein | |
| LigA | A0A381A9U6 |
| Lipoprotein | A0A0E8DGZ8 |
| Lipoteichoic acid synthase 2 | A0A0E7UUG2 |
| LIV-I protein F | A0A0E8EJE1 |
| LIV-I protein H | A0A0E8D3F0 |
| Lon protease | A0A0E7UX91 |
| Long-chain-fatty-acid--CoA ligase | A0A0E8D5M5 |
| LysM domain/BON superfamily | A0A0E8FW56 |
| protein | |
| Major Facilitator Superfamily | A0A381AAV8 |
| Malate dehydrogenase | A0A0E8BHY3 |
| Maltose alpha-D-glucosyltransferase | A0A381A2H0 |
| Manganese transport system | A0A381A652 |
| membrane protein mntB | |
| Membrane dipeptidase (Peptidase | A0A381A4G2 |
| family M19) | |
| Membrane protein insertase YidC | A0A0E8CD07 |
| META domain | A0A380ZXM1 |
| Metal-binding protein | A0A380ZXX5 |
| Methionine--tRNA ligase | A0A380ZXU9 |
| Methyl viologen resistance protein | A0A381A2V9 |
| SmvA | |
| Methylmalonate-semialdehyde | A0A381A7V4 |
| dehydrogenase [acylating] | |
| Mg-chelatase subunit ChlD | A0A381A1P3 |
| MmgE/PrpD family | A0A0E8BVL4 |
| Molybdopterin | A0A381A1C2 |
| molybdenumtransferase | |
| Muconolactone Delta-isomerase | A0A0E7U487 |
| Multidrug export ATP- | A0A0E8CF97 |
| binding/permease protein SAV1866 | |
| Multidrug resistance protein B | A0A381A5U8 |
| Multiple resistance and pH | A0A381A4Q6 |
| homeostasis protein A | |
| Multiple resistance and pH | A0A0E8EGK4 |
| homeostasis protein D | |
| Multiple resistance and pH | A0A381A3P7 |
| homeostasis protein E | |
| N5-carboxyaminoimidazole | A0A381A5C1 |
| ribonucleotide synthase | |
| NAD binding domain of 6- | A0A381A553 |
| phosphogluconate dehydrogenase | |
| NAD(P)H-hydrate epimerase | A0A381A668 |
| NADH-quinone oxidoreductase | A0A381A0E0 |
| NADH-quinone oxidoreductase | A0A0E7UWE6 |
| subunit L | |
| NADH-quinone oxidoreductase | A0A0E8C6U8 |
| subunit M | |
| NADH-quinone oxidoreductase | A0A0E7UKM9 |
| subunit N | |
| NeuSAc permease | A0A380ZZD0 |
| Nickel/cobalt efflux system | A0A381A738 |
| Nicotinamidase/pyrazinamidase | A0A380ZYR7 |
| Nucleoside recognition | A0A381A3G7 |
| Oligopeptidase A | A0A381A1E9 |
| Outer membrane porin protein | A0A0E8D9X0 |
| BP0840 | |
| Outer membrane protein assembly | A0A0E7UA99 |
| factor BamA | |
| Oxoglutarate dehydrogenase | A0A0E8BUQ4 |
| (succinyl-transferring) | |
| P.93 | A0A381A1Z0 |
| Penicillin-binding protein 1A | A0A380ZY64 |
| Peptidyl-prolyl cis-trans isomerase | A0A381A3N0 |
| Peptidyl-prolyl cis-trans isomerase | A0A381A0P3 |
| Peptidyl-prolyl cis-trans isomerase | A0A0E8DEX6 |
| Cbf2 | |
| Periplasmic serine endoprotease | A0A0E7UV64 |
| DegP-like | |
| Pertussis toxin liberation protein F | A0A381A4G6 |
| Phospho-N-acetylmuramoyl- | A0A0E8F538 |
| pentapeptide-transferase | |
| Phosphoserine aminotransferase | A0A0E8EN74 |
| Polyribonucleotide | A0A0E8CDB2 |
| nucleotidyltransferase | |
| Predicted hydrolase of the | A0A0E8F8H1 |
| alpha/beta-hydrolase fold | |
| Predicted membrane protein | A0A381A2P0 |
| Predicted permease, DMT | A0A0E7URC0 |
| superfamily | |
| Predicted protease with the C- | A0A381A875 |
| terminal PDZ domain | |
| Predicted spermidine synthase with | A0A381A530 |
| an N-terminal membrane domain | |
| Preprotein translocase subunit SecD | A0A380ZXS7 |
| Probable ABC transporter ATP- | A0A380ZXW7 |
| binding protein HI_0664 | |
| Probable amino-acid metabolite | A0A0E8F008 |
| efflux pump | |
| Probable chorismate pyruvate-lyase | A0A381A006 |
| Probable lipoate-protein ligase A | A0A381A1P6 |
| Probable membrane transporter | A0A0E8DIF1 |
| protein | |
| Protease HtpX | A0A381A9E0 |
| Protein ApaG | A0A0E8CKK8 |
| Protein HflK | A0A0E7U2I4 |
| Protein of uncharacterized function | A0A380ZY70 |
| (DUF1116) | |
| Protein of uncharacterized function | A0A0E7U6W4 |
| (DUF861) | |
| Protein translocase subunit SecD | A0A0E8DLE5 |
| Protein-L-isoaspartate O- | A0A0E8ECH9 |
| methyltransferase | |
| Purine efflux pump PbuE | A0A381A1J2 |
| Putative aldo-keto reductase | A0A0E8C587 |
| Putative assembly protein | A0A381A6F8 |
| Putative endoribonuclease L-PSP | A0A381A491 |
| Putative glutamate--cysteine ligase 2 | A0A381A797 |
| Putative hydrolase | A0A381A3U8 |
| Putative inner membrane protein | A0A0E7U7L3 |
| Putative NAD(P)H quinone | A0A380ZXI4 |
| oxidoreductase, PIG3 family | |
| Putative protoheme IX biogenesis | A0A381A4J0 |
| protein | |
| Putrescine transport system | A0A0E8EZ60 |
| permease protein PotH | |
| Quinone oxidoreductase 1 | A0A0E7V3C1 |
| Rhodocoxin reductase | A0A381A7C4 |
| Ribonuclease TTHA0252 | A0A0E7U7H7 |
| Ribosomal RNA small subunit | A0A381ABN5 |
| methyltransferase B | |
| Ribosome-binding factor A | A0A0E8DG17 |
| Ribosome-recycling factor | A0A0E8EKA0 |
| RTX-I toxin determinant B | A0A0E8EM13 |
| RutC family protein HI_0719 | A0A0E8CXT3 |
| Sarcosine oxidase subunit beta | A0A0ESDHY6 |
| Sec-independent protein translocase | A0A0E8D1C2 |
| protein TatC | |
| Short-chain fatty acids transporter | A0A0E8CWG2 |
| Single-stranded DNA-binding | A0A380ZWP2 |
| protein | |
| sn-glycerol-3-phosphate transport | A0A0E8D8K9 |
| system permease protein ugpA | |
| Soluble aldose sugar dehydrogenase | A0A381A262 |
| yliI | |
| Soluble lytic murein | A0A381A7I4 |
| transglycosylase | |
| Spermidine/putrescine transport | A0A381A2D5 |
| system permease protein PotB | |
| Sporulation inhibitor kipI | A0A380ZXK7 |
| Stringent starvation protein A | A0A0E8CE82 |
| Succinate dehydrogenase | A0A0E8BE73 |
| flavoprotein subunit | |
| Succinate-semialdehyde | A0A381A059 |
| dehydrogenase [NADP(+)] GabD | |
| Succinate-semialdehyde | A0A0E8CVN1 |
| dehydrogenase [NADP(+)] GabD | |
| Succinate-semialdehyde | A0A0E8D4D1 |
| dehydrogenase [NADP(+)] GabD | |
| Sulfite reductase [ferredoxin] | A0A0E7UJI0 |
| Thiamine biosynthesis protein | A0A381A7A1 |
| HI_0357 | |
| Thiol:disulfide interchange protein | A0A380ZY55 |
| DsbD | |
| Thioredoxin reductase | A0A0E8ENL1 |
| Thiosulfate sulfurtransferase | A0A0E7UKH3 |
| Threonine synthase | A0A0E8E8V7 |
| Thymidylate synthase | A0A0E8BWG3 |
| Tol-Pal system protein TolB | A0A0E8BHZ5 |
| Tol-Pal system protein TolQ | A0A0E8C258 |
| TonB family C-terminal domain | A0A381A5X6 |
| TpsB transporter | A0A0E8BVL7 |
| Transcriptional regulator, y4mF | A0A0E8C4F8 |
| family | |
| Transcription-repair-coupling factor | A0A381A1V7 |
| Transport permease protein | A0A0E8C4R8 |
| TRAP-type C4-dicarboxylate | A0A0E8C7H2 |
| transport system, small permease | |
| component | |
| TRAP-type uncharacterized | A0A381A2Z1 |
| transport system, fused permease | |
| components | |
| Tripartite tricarboxylate transporter | A0A0E8ECZ5 |
| TctA family | |
| tRNA pseudouridine synthase A | A0A381A292 |
| Tryptophan--tRNA ligase | A0A0E7V112 |
| Type IV secretion system protein | A0A381A5L9 |
| virB11 | |
| Tyrosine--tRNA ligase | A0A0E8F3U2 |
| Ubiquinol oxidase subunit 1 | A0A0E7V270 |
| UDP-glucose 6-dehydrogenase | A0A0E7V281 |
| Uncharacterized ACR, COG1565 | A0A381A721 |
| Uncharacterized conserved protein | A0A0E8EFI9 |
| Uncharacterized conserved protein | A0A381A617 |
| Uncharacterized EAL-domain | A0A376C1C7 |
| containing protein ykuI | |
| Uncharacterized FAD-linked | A0A380ZYU0 |
| oxidoreductase Rv2280 | |
| Uncharacterized HTH-type | A0A381A2G2 |
| transcriptional regulator yjiR | |
| Uncharacterized iron-regulated | A0A0E8E188 |
| protein | |
| Uncharacterized oxidoreductase | Q79A65 |
| yvaA | |
| Uncharacterized protein | A0A381A3A0 |
| Uncharacterized protein | A0A381A1L4 |
| Uncharacterized protein | A0A381A2K0 |
| Uncharacterized protein | A0A0E7UN08 |
| Uncharacterized protein | A0A381A328 |
| Uncharacterized protein conserved | A0A380ZXL8 |
| in bacteria | |
| Uncharacterized protein conserved | A0A380ZZ47 |
| in bacteria | |
| Uncharacterized protein conserved | A0A0E8FKG8 |
| in bacteria | |
| Uncharacterized protein conserved | A0A0E8CGX1 |
| in bacteria | |
| Undecaprenyl-diphosphatase | A0A0E7U5B1 |
| Universal stress protein | A0A0E8G816 |
| Rv1996/MT2052 | |
| Universal stress protein SAV1710 | A0A0E8BUB2 |
| UPF0250 protein | A0A0E8CCN8 |
| NCTC10911 00447 | |
| Vi polysaccharide biosynthesis | A0A381A4X9 |
| protein TviD | |
| Virulence-associated outer | A0A0E8DWP6 |
| membrane protein Vir-90 | |
| Xaa-Pro aminopeptidase | A0A381A0H0 |
| Xanthine dehydrogenase accessory | A0A381A311 |
| protein XdhC | |

SEQUENCES

1. SEQ ID NO: 1-(full length BP020S sequence)
MQLTIRKLAYTLAFSTLVLAGCTTASKKTDGQAATPADQASSQQASAAS
VEFYVAQAKAGDGLMEVKVPDGSLYMQRQPVLTRADLTEAAALVDRQGQ
NFVGLRFTEAGARKLNDISSKNIGNMLALVIDRELVAAPRIAEPLNRGV
LAFGVPSAKAASEIAAKIRGDAGAPAAGVPAAPAPKPAPK
2. SEQ ID NO: 2-(Antigen epitope)
IDRELVAAPRIAEPL
3. SEQ ID NO: 3-(Antigen epitope)
GVLAFGVPSAKAASEI
4. SEQ ID NO: 4-(Signal peptide)
MQLTIRKLAYTLAFSTLVLAGC

1. A pertussis vaccine comprising a BP0205 antigen in combination with at least one additional peptide from a Bordetella pertussis (B. pertussis) bacterium, wherein the BP0205 peptide and the at least one additional peptide are to be presented to an MHC Class II cell and recognized by a CD4+ T cell.

2. (canceled)

3. (canceled)

4. The vaccine of claim 1, wherein the BP0205 antigen comprises at least 90% sequence identity to SEQ ID NO: 1.

5. The vaccine of claim 1, wherein the BP0205 antigen comprises SEQ ID NO: 1.

6. The vaccine of claim 1, wherein the vaccine comprises an antigen epitope comprising SEQ ID NO: 2 and/or SEQ ID NO: 3.

7. (canceled)

8. The vaccine of claim 1, wherein the at least one peptide originates from a protein involved in at least one biological pathway within the B. pertussis bacterium.

9. The vaccine of claim 8, wherein the at least one pathway is selected from cell adhesion and motility, cell cycle, cell signaling, cell structure, cell transport, gene regulation, metabolism, oxidation-reduction, protein regulation, or stress responses.

10-19. (canceled)

20. The vaccine of claim 1, wherein the pertussis vaccine further comprises any combination of adjuvants, preservatives, or stabilizers.

21. The vaccine of claim 1, wherein the vaccine comprises a pharmaceutically acceptable carrier selected from an excipient, a diluent, a salt, a buffer, a stabilizer, a lipid, an emulsion, or a nanoparticle.

22. A method of preventing an infection from a Bordetella pertussis (B. pertussis) bacterium in a subject, the method comprising administering to the subject a pharmaceutically effective amount of the pertussis vaccine of claim 1.

23. A method of improving an immune response against a Bordetella pertussis (B. pertussis) bacterium in a subject, the method comprising administering to the subject a pharmaceutically effective amount of the pertussis vaccine of claim 1.

24. The method of claim 22, wherein the infection from the Bordetella pertussis bacterium causes whooping cough in the subject.

25. The method of claim 22, wherein the immune response comprises presenting the antigen to an MHC Class II cell, recognition of the antigen by a CD4+ T cell, or combination thereof.

26. The method of claim 22, wherein the immune response is against a BP0205 antigen and at least one protein involved in at least one biological pathway within the B. pertussis bacterium.

27. The method of claim 26, wherein the at least one pathway selected from cell adhesion and motility, cell cycle, cell signaling, cell structure, cell transport, gene regulation, metabolism, oxidation-reduction, protein regulation, or stress responses.

28. The method of claim 22, wherein the method further comprises administering the pertussis vaccine through intranasal, intramuscular, intraperitoneal, or subcutaneous routes of injection.

29. The method of claim 22, wherein the subject is a mammal.

30. The method of claim 22, wherein the subject is a human.
